(19) United States
(12) Patent Application Publication
(10) Pub. No.: US 2006/0193893 A1
(43) Pub. Date: Aug. 31, 2006

(54) Medical devices

(75) Inventor: Dennis M. Brown, Menlo Park, CA (US)

Correspondence Address: Richard F. Trecartin;Dorsey & Whitney LLP, Intellectual Property Department, 555 California Street, 10th Floor, San Francisco, CA, 94104-1513, US

(73) Assignee: ChemGenex Pharmaceuticals, Inc.

(21) Appl. No.: 11/351,510

(22) Filed: Feb. 10, 2006

Related U.S. Application Data
(60) Provisional application No. 60/651,757, filed on Feb. 10, 2005.

(51) Int. Cl. A61K 31/727; A61F 2/06; A61K 31/55

(52) U.S. Cl. 424/426; 514/56; 514/510; 514/214.03; 623/1.11

(57) ABSTRACT

The invention relates to medical devices, compositions and methods useful for administrating or delivering a cephalotaxine to a host. The cephalotaxines are useful in treating or preventing an angiogenic condition or disease. The invention provides for medical devices including coatings containing a matrix and a cephtalotaxine to treat an angiogenic condition or disease. The invention provides for methods and compositions, including cephalotaxine alkaloids as antiangiogenic agents, for treatment of a host with an angiogenic condition or disease or for prophylactic treatment of a host to inhibit the onset or progression of an angiogenic condition or disease. The invention also provides for medical devices useful for delivering or administrating a cephalotaxine in vivo.

This application claims the benefit of U.S. Ser. No. 60/651,757, filed Feb. 10, 2005.

### FIELD OF THE INVENTION

The invention relates to medical devices, compositions and methods useful for administrating or delivering cephalotaxine to a host.

### BACKGROUND OF THE INVENTION

Angiogenesis is defined as the formation and differentiation of new blood vessels. It has been linked to a number of diseases and conditions, in particular to cancer, inflammation and certain retinal disorders. Angiogenesis inhibitors have recently become high profile agents in the fight against cancer, with several compounds, most notably angiostatin, endostatin, combretastatin, SU5416, TNP470, anti-VEGF compounds and others, have advanced into clinical trials as anticancer agents.

Angiogenesis, the process by which new blood vessels are formed, is essential for normal body activities including reproduction, development, and wound repair. Although the process is not completely understood, it is believed to involve a complex interplay of molecules that regulate the growth of endothelial cells (the primary cells of capillary blood vessels). Under normal conditions, these molecules appear to maintain the microvasculature in a quiescent state (i.e. one of no capillary growth) for prolonged periods which may last for as long as weeks, or, in some cases, decades. When necessary (such as during wound repair), these same cells can undergo rapid proliferation and turnover within a 5 day period (Folkman, J. and Shing, Y.; J. Biol. Chem., 267 (16), 10931-10934, and Folkman, J. and Klagsbrun, M. Science, 235, 442-447 (1987).

Although angiogenesis is a highly regulated process under normal conditions, many diseases (characterized as angiogenic diseases or conditions) are driven by persistent unregulated angiogenesis. Otherwise stated, unregulated angiogenesis may either cause a particular disease directly or exacerbate an existing pathological condition. For example, ocular neovacularization has been implicated as the most common cause of blindness and dominates approximately 20 eye diseases. In certain existing conditions, such as arthritis, newly formed capillary blood vessels invade the joints and destroy cartilage. In diabetes, new capillaries formed in the retina invade the vitreous, bleed, and cause blindness. Growth and metastasis of solid tumors are also dependent on angiogenesis (Folkman, J., (1986) Cancer Research, 46, 467-473, Folkman, J., (1989) J. National Cancer Institute, 82, 4-6, both of which are hereby expressly incorporated by reference). It has been shown, for example, that tumors that enlarge greater than 2 mm must obtain their own blood supply and do so by inducing the growth of new capillary blood vessels. Once these blood vessels become embedded in the tumor, they provide a means for the tumor to metastasize to different sites such as liver, lung or bone (Weidner, N. et al., (1991) The New England Joumal of Medicine, 324(1), 1-8).

To date, several naturally occurring angiogenic factors have been described and characterized (Fidler, J., I. and Ellis, L. M., (1994) Cell, 79, 185-189). Recently, O'Reilly, et al. have isolated and purified a 38 kilodalton (kDa) protein from serum and urine of tumor-bearing mice that inhibits endothelial cell proliferation (O'Reilly, M et al., (1994) Cell, 79, 315-328 and International Application WO 95/29242, published Nov. 2, 1995). Microsequence analysis of this endothelial inhibitor showed 98% sequence homology to an internal fragment of murine plasminogen. Angiostatin, as the murine inhibitory fragment was named, was a peptide that included the first four kringle regions of murine plasminogen. A peptide fragment from the same region of human plasminogen (i.e. containing kringles 1-4) also strongly inhibited proliferation of capillary endothelial cells in vitro and in vivo. The intact plasminogen from which this peptide fragment was derived did not possess as potent an inhibitory effect.

Several angiogenesis inhibitors are currently under development for use in treating angiogenic diseases (Gasparini, G. and Harris, A. L., (1995) J. Clin. Oncol., 13(3): 765-782), but there are disadvantages associated with these compounds. Suramin, for example, is a potent angiogenesis inhibitor but causes severe systemic toxicity at the doses required for antitumor activity. Compounds such as retinoids, interferons and antiestrogens are safe for human use but have weak antiangiogenic effects.

Arterial narrowing and blood clotting are two related, life-threatening conditions commonly associated with the cardiovascular system. Stenosis is the narrowing of a blood vessel, usually due to fat and/or cholesterol buildup. Thromobosis is the formation of a blood clot inside a vessel or cavity of the heart. Both can cause vascular obstruction. Currently, an approach to clogged or constricted arteries due to stenosis is balloon angioplasty, or percutaneous transluminal coronary angioplasty (PTCA). However, approximately 30% to 40% of patients who undergo PTCA suffer restenosis or a renarrowing of the vessel within 3 to 6 months of the procedure. Restenosis primarily results from the proliferation of vascular smooth muscle cells and extracellular matrix secretion at the site of injury. Such patients may have to undergo a subsequent angioplasty. Restenosis can be inhibited through the use of a medical device such as a stent, which can buttress the artery that has recently been widened through angioplasty to prevent elastic recoil of the artery. Non-coronary blood vessels are likewise affected by restenosis. The carotid, femoral, iliac, and renal arteries may be subject to a renarrowing following an angioplasty and/or stent procedure.

Recent studies have suggested that the stent-based delivery of the angiogenesis inhibitor, angiostatin, may provide a beneficial effect to patients following angioplasty and stent placement. (Ganaha F. et al., J. Vasc. Interv. Radiol. 2004 June;15(6):601-8). In addition, multi-coated drug-eluting stents for anti-thrombotic and anti-restenosis therapies have been developed. (Byun et al., U.S. Pat. No. 6,702,850 and Yang et al., U.S. Pat. No. 6,258,121)

Thus, there is a need for compounds useful in treating angiogenic diseases or conditions in mammals. Additionally, there is a need for compounds useful in the prophylactic treatment of a host to prevent or inhibit the onset, progression or reoccurrence of angiogenic diseases or conditions. Furthermore, there is a need for medical devices capable of delivering angiogenesis inhibitors to patients having an angiogenic disease or condition and/or undergoing a medical procedure, such as PTCA.

While several antiangiogenic inhibitors have been identified, improvements in clinical use are still sought. The invention described herein demonstrates medical devices and novel uses thereof of the cephalotaxine alkaloids and derivatives including homoharringtonine that can inhibit angiogenesis and thereby affect angiogenic diseases or conditions.

### SUMMARY OF THE INVENTION

The invention relates to medical devices, compositions and methods useful for administrating or delivering cephalotaxine to a host. The cephalotaxines are useful in treating or preventing an angiogenic condition or disease. The invention provides for medical devices including coatings containing a matrix and a cephtalotaxine to treat an angiogenic condition or disease. The invention provides for methods and compositions, including cephalotaxine alkaloids as antiangiogenic agents, for treatment of a host with an angiogenic condition or disease or for prophylactic treatment of a host to inhibit the onset or progression of an angiogenic condition or disease. The invention also provides for medical devices useful for delivering or administrating a cephalotaxine in vivo.

In one embodiment, a medical device has a device body and a coating (matrix) on a surface of the device body where the coating includes a cephalotaxine alone or in combination with another active agent. Such devices may be catheters, endoscopes, wound healing dressings, tissue/organ barriers, sutures, artificial organs, artificial organoids, implantable monitors, defibrillators, pacemakers, implantable pumps, cell reservoirs, prosthetic devices, and orthopedic devices. In some embodiments, the device is other than a stent.

In another embodiment, the medical device is composed of a stent and a coating (matrix) on a surface of the stent that contains a cephalotaxine. In some embodiments, the stent does not have an additional single heparin-containing coating. The coatings of the devices of the present invention may include polymers, such as a biopolymer or a synthetic polymer. Alternatively, the coatings may include non-polymeric materials. The cephalotaxines contained in the coatings include homoharringtonine (cephalotaxine, 4-methyl-2-hydroxy-2-(4-hydroxy-4-methyl pentyl) butanediocate ester) or a cephalotaxine analog. The medical devices may include a more than one coating and the coatings may include a second agent.

The medical device can also be a matrix containing cephalotaxine alone or in combination with other active agents.

Such medical devices may be employed for applications in cardiology, ophthalmology, inflammatory disease, infection control, surgical adhesion control, intraoperative applications, wound healing management, burn dressings, medical imaging, and the like.

The present invention also provides a method of contacting a host with a medical device in vivo. In some embodiments, the device provides a sufficient amount of cephalotaxine to inhibit angiogenesis. In other embodiments, the cephalotaxine inhibits the onset or progression of an angiogenic disease. The medical device is preferably implanted into the host. The invention also provides a delivery device having a catheter with a lumen and a cephalotaxine containing a stent contained within the lumen.

### BRIEF DESCRIPTION OF THE DRAWINGS

FIG. 1 depicts the general chemical structure of the cephalotaxine family.

FIG. 2 depicts the chemical structure of homoharringtonine.

FIG. 3 depicts chorioallantoic membrane (CAM) vessels.

FIG. 3B depicts the veins within the CAM.

FIG. 3C depicts the veins and arteries within the CAM.

FIG. 4 depicts effects of homoharringtonine in the CAM.

FIG. 5 depicts the comparison between qualitative changes caused by homoharringtonine and taxol.

### DETAILED DESCRIPTION

The embodiments of the invention described herein are not intended to be exhaustive or limit the invention to those specifically disclosed below. The disclosed embodiments have been selected so that a person of ordinary skill in the art can appreciate and understand the principles and practice of the present invention.

Medical devices, methods and compositions are provided for the delivery or administration of cephalotaxines to a host to treat a disease or condition. Cephalotaxines may be useful in the (1) treatment of a host with an angiogenic disease, and the (2) prophylactic treatment of a host to prevent the onset or progression of an angiogenic disease. In some embodiments, the cephalotaxine is used to treat a disease or condition in a host by having an effect on the host, including without limitation, the inhibition of angiogenesis, the inhibition of inflammation, and/or the inhibition of cell proliferation.

The medical devices, methods and compositions provided herein are used in or the treatment of a host. A “host” includes both humans and other animals, particularly mammals. Thus the methods are applicable to both human therapy and veterinary applications. The host may be a patient. In the preferred embodiment the patient is a mammal, and in the most preferred embodiment the patient is human.

A compound or chemical agent, such as a cephalotaxine are known for their antiproliferation properties. Cephalotaxines are used herein, in preferred embodiments, as an inhibitor of angiogenesis, i.e., an inhibitor of blood vessel formation. Cephalotaxines are alkaloids extracted from skins, stems, leaves and seeds of Cephalotaxus fortunei Hook and other related species, such as Cepholotaxus sinensis Li, C. hainanensis and C. wilsoniana, including C. oliveri mast and C. harringtonia (Powell, R. G., (1972) J. Pharm Sci., 61(8):1227-1230).

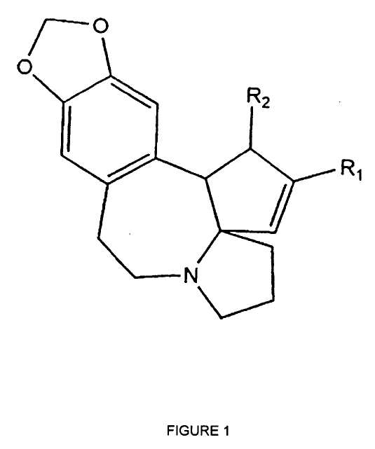

As used herein, the term cephalotaxine includes all members of that chemical family including alkaloid derivatives of the Chinese evergreen, Cephalotaxus fortunei and analogs thereof. The cephalotaxine family is defined by chemical structure as set forth in FIG. 1.

A cephalotaxine analog is defined but not limited to the structure depicted in FIG. 1, having substituent or substitute groups at R₁ and R₂. Examples of R₁ and/or R₂ include esters that form harringtonine, isoharringtonine, homoharringtonine, deoxyharringtonine, acetylcephalotaxine and the like. Table 1 lists structures of R₁ and R₂ for some of these analogs. R₁ and R₂ substitutions are typically employed to improve biological activity, pharmaceutical attributes such as bioavailability or stability, or decreased toxicity. In one embodiment, R₁ and/or R₂ include alkyl substitutions (e.g., methyl, ethyl, propyl etc.). In another embodiment, R₁ and/or R₂ include ethers (e.g. methoxy, ethoxy, butoxy, etc.). In other embodiments, R₁ and/or R₂ are esters, e.g., —(O—C(O)—X), where X is an alkyl or a substituted alkyl.

TABLE 1

| | R₁ | R₂ |
|---|---|---|
| isoharringtonine | —OCH₃ | 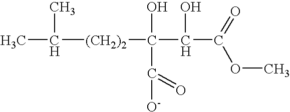 |
| harringtonine | —OCH₃ | 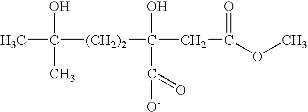 |
| acetylcephalotaxine | —OCH₃ | 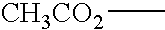 |
| homoharringtonine | —OCH₃ | 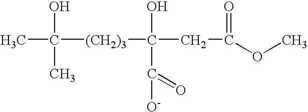 |

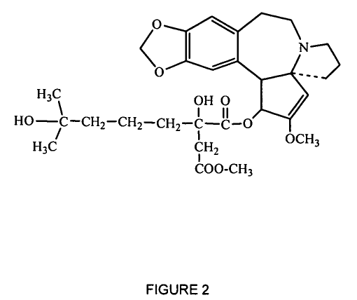

A specific example of cephalotaxine is homoharringtonine which is the butanediocate ester of cephalotaxine, 4-methyl-2-hydroxy-2-(4-hydroxy-4-methyl pentyl) (FIG. 2).

As illustrated in the Examples, cephalotaxines are angiogenic inhibitors. It is an aspect of the invention to provide medical devices, methods, and compositions for the delivery or the administration of cephalotaxine to a host. In each case, the cephalotaxine is given in an amount sufficient to inhibit or prevent angiogenesis. In preferred embodiments, the cephalotaxine is used because of its anti-angiogenic properties, i.e., to treat an angiogenic disease. In a further aspect, the invention provides medical devices, methods, and compositions for delivery of a cephalotaxine for use in prophylactic treatments to prevent the onset or progression of an angiogenic disease or condition.

The inhibition of angiogenesis by cephalotaxine is useful in treating or preventing an angiogenic disease or condition. Examples of angiogenic diseases or conditions include, but are not limited to, diabetic retinopathy, inflammatory diseases (such as rheumatoid arthritis, osteoarthritis, asthma, and pulmonary fibrosis), macular degeneration, angiofibroma, neovascular glaucoma , arteriovenous malformations, nonunion fractures, lupus and other connective tissue disorders, Osler-Weber syndrome, atherosclerotic plaques, stenosis, thrombosis, restenosis, psoriasis, corneal graft neovascularization, pyogenic granuloma, retrolental fibroplasia, scleroderma , granulations, hemangioma , trachoma , hemophilic joints, and vascular adhesions.

In some embodiments, the medical device is used to deliver cephalotaxine to a solid tumor to inhibit angiogenesis. In this case, solid tumor function is combined with an angiogenic disease. Growth and metastasis of tumors is dependent on angiogenesis. Solid tumors need oxygen and nutrients to survive and grow. Without a blood supply, potential tumors either die or remain dormant. These potential tumors can be, for example, microtumors or micrometastatic cancer cells. The “microtumors” remain as a stable cell population wherein dying cells are replaced by new cells. Microtumors may represent, for example, the initiation of a solid tumor in host that has no other solid tumors. Microtumors may also represent the remaining tumor cells present in a host after the solid tumor, from which the microtumors has metastasized, has been removed or eradicated. This condition may occur in a host that is in remission for cancerous tumors. Micrometastatic cancer cells refers to cancer cells that have not yet been vascularized to form a solid tumor.

The microtumor becomes a rapidly growing tumor when it becomes vascularized and can expand to 16,000 times its original volume in 2 weeks after vascularization. Without the blood supply, no growth is seen (Folkman, J. (1974) Tumor Angiogenesis, Adv. Cancer Res. 19: 331 358; Ausprunk, D. H. and Folkman, J. (1977) Migration and Proliferation of Endothelial Cells in Preformed and Newly Formed Blood Vessels During Tumor Angiogenesis, Microvasc. Res. 14: 53 65, both of which are hereby expressly incorporated by reference).

In addition to supplying the tumor with nutrients and oxygen, angiogenesis allows the solid tumor to metastasize. The new blood vessels provide a route that enables cells from the solid tumor to migrate to other sites in the host, resulting in the formation of secondary tumors.

Thus, by inhibiting angiogenesis, the vascularization of tumors and/or microtumors is minimized and the progression of metastasis and tumor growth is inhibited or stopped.

In one embodiment of the invention, a cephalotaxine is administered or delivered to a host with microtumors. The cephalotaxine is administered or delivered in an amount sufficient to inhibit angiogenesis thereby inhibiting growth and metastasis of the microtumors. The microtumors may represent the early onset of a disease characterized by tumor growth. The microtumors may be the result of metastasis of an established solid tumor.

Another disease characterized by excessive blood vessel growth is diabetic retinopathy. Recent studies indicate a pathogenetic role for the renin-angiotensin system (RAS) and vascular endothelial growth factor (VEGF) in the eye in response to chronic hyperglycaemia (Wilkinson-Berka J. L., et al., (2001) The Interaction Between the Renin-Angiotensin System and Vascular Endothelial Growth Factor in the Pathogenesis of Retinal Neovascularization in Diabetes, J Vasc Res., 38(6):527-35).

In one embodiment of the invention, a cephalotaxine is administered to a host suffering from, or at the risk of suffering from, diabetic retinopathy. The cephalotaxine is administered or delivered in an amount sufficient to inhibit angiogenesis thereby slowing progression of the diabetic retinopathy. In a preferred embodiment, a medical device comprises matrix particles containing cephaloxtaxines are intraoccularly administered so as to be dispersed at or near the retina.

Angiogenesis has been implicated in chronic inflammatory diseases, including for example, rheumatoid arthritis, osteoarthritis, asthma, and pulmonary fibrosis (Walsh, D. A. and Pearson, C. I. (2001), Angiogenesis in the Pathogenesis of Inflammatory Joint and Lung Diseases, Arthritis Res., (3): 147-153; Storgard1, C. M., et al., (1999), Decreased Angiogenesis and Arthritic Disease in Rabbits Treated with an vβ3 Antagonist, J Clin Invest, 3(1):47-54, each of which is expressly incorporated by reference).

In one embodiment of the invention, a cephalotaxine is administered or delivered to a host with an inflammatory disease in an amount sufficient to inhibit angiogenesis thereby slowing progression of the inflammatory disease. In a preferred embodiment of the invention, the inflammatory disease is rheumatoid arthritis. In a further preferred embodiment, the inflammatory disease is osteoarthritis. In each case of arthritis, medical devices comprising a matrix containing cephalotaxines are injected into the bursa of an affected joint. In another embodiment, the inflammatory disease is asthma or pulmonary fibrosis.

The cephalotaxine can be administered or delivered to a host as a prophylactic treatment. By “prophylactic treatment” is meant administration or delivery of a cephalotaxine to a host to prevent the onset or progression of an angiogenic disease. In this embodiment of the invention, a cephalotaxine is administered or delivered to a host to prevent the onset of tumor growth or metastasis or a disease characterized by tumor growth or metastasis. Such treatment may be desirable, for example, in a host that has exhibited tumor growth, such as a cancerous tumor, but is now in remission.

The cephalotaxine can also be administered or delivered to a host to prevent the onset or progression of an angiogenic disease other than cancerous tumor growth. In this embodiment, the cephalotaxine is administered or delivered to a host at risk of exhibiting an inflammatory disease, such as rheumatoid arthritis, osteoarthritis, asthma, or pulmonary fibrosis.

In a further preferred embodiment, a cephalotaxine is administered or delivered to a host that is diabetic, or at risk of becoming diabetic, as a prophylactic treatment to prevent or inhibit the onset of diabetic retinopathy. In yet a further preferred embodiment, cephalotaxine is administered or delivered to a host that is at risk of exhibiting macular degeneration (such as an elderly human) as a prophylactic treatment to prevent or inhibit the onset of macular degeneration.

For the prophylactic treatments above, the cephalotaxine is administered or delivered in amount sufficient to inhibit the onset or progression of the angiogenic disease.

The medical devices can be (1) a device body coated with a matrix comprising cephalotaxines alone or in combination with other active agents or (2) a matrix containing cephalotaxines alone or in combination with other active agents. The medical devices can be delivered orally, intravenously, topically, intravescularly, intraperitoneally, intramuscularly or intradermally. The matrix may be a biodegradable polymer allowing for sustained release of the cephalotaxine. Such biodegradable polymers are described, for example, in detail in Brem et al., J. Neurosurg. 74:441-446 (1991) and elsewhere herein.

The types of active agents or drugs other than cephaloxtaine that may be employed within the matrices of the medical devices include antiproliferative agents, cytostatic agents, cytotoxic agents, apoptosis inducers, signal transduction affectors, kinase and phosphatase inhibitors and inducers, radiation sensitiziers, radiation protectors, DNA repair inhibitors, antiviral agent, antibacterial agents antifungal agents antiparasitic agents, cancer chemotherapeutic agents, anti-inflammatory agents, compounds affecting lipid metabolism, compounds affecting glucose, neuroactive/neuroprotective agents,drug resistance reversal agents, chemoprotective agents, cytokines, growth factors, lymphokines, therapeutic antibodies, gene therapies RNAi/antisense therapeutics and the like.

In some embodiments, the active agents suitable for use in a matrix include without limitation compounds of natural product origin, plant alkaloids, macrolides, terpenes, antibiotics, vinca alkaloids, camptothecins, taxanes, taxane analogs, bruceantin, vancomycins, and the like. For example, cephalotaxines, as described herein, are active as antiproliferative agents. As described herein, they also affect angiogenesis, induce apoptosis, and affect aspects of signal transduction pathways would be employed with matrices described herein alone or in combination with the other agents and/or excipients described herein to create novel useful materials such as medical devices or therapeutic materials.

In one embodiment, the active agents suitable for use in a matrix may include without limitation anti-cancer drugs acivicin, aclarubicin, acodazole, acronycine, adozelesin, alanosine, aldesleukin, allopurinol sodium, altretamine, aminoglutethimide, amonafide, ampligen, amsacrine, androgens, anguidine, aphidicolin glycinate, asaley, asparaginase, 5-azacitidine, azathioprine, Bacillus calmette-guerin (BCG), Baker's Antifol (soluble), beta-2′-deoxythioguanosine, bisantrene hcl, bleomycin sulfate, busulfan, buthionine sulfoximine, BWA 773U82, BW 502U83.HCl, BW 7U85 mesylate, ceracemide, carbetimer, carboplatin, carmustine, chlorambucil, chloroquinoxaline-sulfonamide, chlorozotocin, chromomycin A3, cisplatin, cladribine, corticosteroids, Corynebacterium parvum, CPT-11, crisnatol, cyclocytidine, cyclophosphamide, cytarabine, cytembena, dabis maleate, dacarbazine, dactinomycin, daunorubicin HCl, deazauridine, dexrazoxane, dianhydrogalactitol, diaziquone, dibromodulcitol, didemnin B, diethyldithiocarbamate, diglycoaldehyde, dihydro-5-azacytidine, doxorubicin, echinomycin, edatrexate, edelfosine, eflomithine, Elliott's solution, elsamitrucin, epirubicin, esorubicin, estramustine phosphate, estrogens, etanidazole, ethiofos, etoposide, fadrazole, fazarabine, fenretinide, filgrastim, finasteride, flavone acetic acid, floxuridine, fludarabine phosphate, 5-fluorouracil, Fluosol.RTM., flutamide, gallium nitrate, gemcitabine, goserelin acetate, hepsulfam, hexamethylene bisacetamide, homoharringtonine, hydrazine sulfate, 4-hydroxyandrostenedione, hydrozyurea, idarubicin HCI, ifosfamide, interferon alfa, interferon beta, interferon gamma, interleukin-1 alpha and beta, interleukin-3, interleukin-4, interleukin-6, 4-ipomeanol, iproplatin, isotretinoin, leucovorin calcium, leuprolide acetate, levamisole, liposomal daunorubicin, liposome encapsulated doxorubicin, lomustine, lonidamine, maytansine, mechlorethamine hydrochloride, melphalan, menogaril, merbarone, 6-mercaptopurine, mesna, methanol extraction residue of Bacillus calmette-guerin, methotrexate, N-methylformamide, mifepristone, mitoguazone, mitomycin-C, mitotane, mitoxantrone hydrochloride, monocyte/macrophage colony-stimulating factor, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, PIXY-321, plicamycin, porfimer sodium, prednimustine, procarbazine, progestins, pyrazofurin, razoxane, sargramostim, semustine, spirogermanium, spiromustine, streptonigrin, streptozocin, sulofenur, suramin sodium, tamoxifen, taxotere, tegafur, teniposide, terephthalamidine, teroxirone, thioguanine, thiotepa, thymidine injection, tiazofurin, topotecan, toremifene, tretinoin, trifluoperazine hydrochloride, trifluridine, trimetrexate, tumor necrosis factor, uracil mustard, vinblastine sulfate, vincristine sulfate, vindesine, vinorelbine, vinzolidine, Yoshi 864, zorubicin, and mixtures thereof.

In another embodiment, the active agent is an anti-inflammatory drugs, which may include without limitation, non-steroidal anti-inflammatory drugs (NSAIDS), such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen (Tylenol.RTM.), and mixtures thereof; COX-2 inhibitors, such as nimesulide, NS-398, flosulid, L-745337, celecoxib, rofecoxib, SC-57666, DuP-697, parecoxib sodium, JTE-522, valdecoxib, SC-58125, etoncoxib, RS-57067, L-748780, L-761066, APHS, etodolac, meloxicam, S-2474, and mixtures thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, and mixtures thereof; and mixtures thereof.

In one embodiment, the active agent may be an medical imaging agent including without limitation paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in any fabrication method or absorbed into or sprayed onto the surface of part or all of a medical device as described herein. Radiopacifiers (such as radio opaque materials) can be included in any fabrication method or absorbed into or sprayed onto the surface of part or all of a medical device of the present invention. The degree of radiopacity contrast can be altered by controlling the concentration of the radiopacifier within or on the implant. Radiopacity can be imparted by covalently binding iodine to the polymer monomeric building blocks of the elements of the implant. Common radio opaque materials include barium sulfate, bismuth subcarbonate, and zirconium dioxide. Other radio opaque materials include cadmium, tungsten, gold, tantalum, bismuth, platinum, iridium, and rhodium. In some embodiments, iodine can be employed for both its radiopacity and antimicrobial properties. This can be useful for detection of medical devices described herein that are implanted in the body (that are emplaced at the treatment site) or that travel through a portion of the body (that is, during implantation of the device). Paramagnetic resonance imaging, ultrasonic imaging, x-ray means, fluoroscopy, or other suitable detection techniques can detect medical devices including these materials. In some embodiments, the medical imaging agent may assist in medical imaging of a medical device described herein once implanted.

In another embodiment, the active agent may be an immunosuppressive agent including without limitation a cyclosporin, tacrolimus FK506, rapamycin (sirolimus), and analogues of rapamycin. Analogs of rapamycin include without limitation CCl-779 (Wyeth), RAD001 or everolimus (Novartis), and AP23573 (Ariad Pharmaceuticals). Rapamycin can be used to prevent renal transplant rejection. It has also been reported to be effective in preventing restenosis. (Serruys, P. E. et al., Heart 2002 87;305-307.) In one embodiment, a matrix includes a cephalotaxine and rapamycin or one of its analogs. In another embodiment, a matrix includes a cephalotaxine, rapamycin or one of its analogs, and another active agent.

In another embodiment, the active agent may a promoter of wound healing, including without limitation granulocyte-macrophage colony-stimulating factor (GM-CSF) and/or a growth factor.

In another embodiment, a matrix containing a first active agent and a second active agent are delivered to a host directly or by a medical device body coated with the matrix such that the amount of the first active agent delivered modulates a condition or disease in the host. The amount of the first active agent delivered may be such that the modulation achieved is greater than it would have been if the host had received the second active agent absent the first active agent. In a preferred embodiment, the first active agent is a cephalotaxine. In a preferred embodiment, the medical device is a stent. In another preferred embodiment, the condition modulated is restenosis. In a more preferred embodiment, the modulation is an inhibition of restenosis.

The active agents or drugs may be either small chemical structures from natural or synthetic sources, radionuclides or biologicals such as therapeutic peptides/ proteins, nucleic acid polymers such as DNA, mammalian sequences or non-mammalian such as plasmids or viral or synthetic nucleic acid polymers such as antisense nucleic acids or interference RNA are contained alone or in varying combinations and concentrations to create the appropriate biological/disease management effects. The active agents contained within the biologically tolerable matrices maybe linked or non-linked to the matrices. Linkage may be created through covalent, ionic, hydrogen bonds through complexation or entrapment. Particles of varying size of the active agent(s) may be employed include nanoparticles, microparticles, emulsions with or without surfactants and stabilizers. In addition, other agents and excipients may be employed to provide controlled release, enhanced stability, antioxidation, etc.

Devices that may employ these matrices can include without limitation catheters (balloon or inflation catheters, injection catheters, central venous catheters, and arterial catheters), stents (vascular stents, urethra stents, bile duct stents, biliary stents, esophageal stents, tracheal or bronchial stents), vascular stent grafts, endoscopes, wound healing dressings, tissue barriers or organ barriers (e.g., surgical adhesion prevention), sutures, artificial organs or artificial organoids (e.g., insulin secreting device), implantable monitors, defibrillators, ventricular assist devices, pacemakers, implantable pumps, cell reservoirs (e.g., for stem cell placement), prosthetic devices including prosthetic heart valves, orthopedic devices. Other devices include without limitation surgical staples, guidewires, cannulas, cardiac pacemaker and electrostimulation leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, septal defect devices, hemodialysis units, hemoperfusion units, plasmapheresis units, anastomosis devices, implantable biosensors, implanted drug infusion tubes, birth control occlusion devices, breast implants, pain management devices, prostate cancer treatment devices, dental implants, focal epilepsy treatment devices, nerve regeneration conduits, vena cava filters, spinal repair devices, spinal cord stimulators, internal hearing aids, neuro aneurysm treatment devices, heart valve repair devices, intravitreal drug delivery devices, joint replacements, ophthalamic implants, needles, and vascular grafts.

Medical devices suitable for the present invention include those that have a tubular or cylindrical-like portion. In another embodiment, the device is in the form of a disc. The disc may be composed of stainless steel or another biocompatible material as described herein. The tubular portion of the medical device need not be completely cylindrical. For instance, the cross-section of the tubular portion can be any shape, such as rectangle, a triangle, etc., not just a circle. Such devices include, without limitation, stents, balloon catheters, and grafts. A bifurcated stent is also included among the medical devices which can be fabricated according to the present invention. Medical devices that are particularly suitable for the present invention include any kind of stent for medical purposes which is known to the skilled artisan.

The devices of the present invention may be composed in part or entirely of biocompatible materials, which typically have the ability to support a tissue. In one embodiment, the tissue is a blood vessel, preferably a defective blood vessel. In one embodiment, the material is a biocompatible metallic material. The metallic material may be a metal or an alloy. The types of metallic material include without limitation titanium, nitinol, nickel titanium alloys, thermo-memory alloy materials, stainless steel, tantalum, nickel-chrome, gold and certain cobalt alloys including cobalt-chromium-nickel alloys.

In another embodiment, the biocompatible material is plastic, ceramic, or another appropriate material. Ceramic materials may include without limitation oxides, carbides, or nitrides of the transition elements such as titanium oxides, hafnium oxides, iridium oxides, chromium oxides, aluminum oxides, and zirconium oxides. Silicon based materials, such as silica, may also be used.

In one embodiment, the present invention provides medical devices suitable for in vivo use in a patient. Such a use may include implantation into a patient. The medical device may be composed in part or entirely of a biodegradable or bioabsorbable material. In another embodiment, the medical device is an implantable intraluminal device. A biologically active material may be delivered to a body lumen using a medical device described herein. For example, a stent may be inserted into body of the patient by a method known to a person of ordinary skill. When the stent is a self-expandable stent, it can be collapsed to a small diameter by placing it in a sheath, introduced into a lumen of a patient's body using a catheter, and allowed to expand in the target area by removing it from the sheath. When the stent is a balloon expandable stent, it may be collapsed to a small diameter, placed over an angioplasty balloon catheter, and moved into the area to be placed. When the balloon is inflated, the stent expands.

The devices of the present invention, such as a stent, may be utilized in connection with an expandable intraluminal vascular graft for expanding partially occluded segments of a vessel, duct, body passageway, or duct, such as within an organ. In addition, such a device may also be utilized for many other purposes as an expandable prosthesis for many other types of body passageways. For example, expandable prostheses can also be used for such purposes as (1) supportive graft placement within blocked arteries opened by transluminal recanalization having the potential to collapse in the absence of internal support; (2) similar use following catheter passage through mediastinal and other veins occluded by inoperable cancers; (3) reinforcement of catheter created intrahepatic communications between portal and hepatic veins in patients suffering from portal hypertension; (4) supportive graft placement of narrowing of the esophagus, the intestine, the ureters, the urethra, and the like; (5) intraluminally bypassing a defect such as an aneurysm or blockage within a vessel or organ; and (6) supportive graft reinforcement of reopened and previously obstructed bile ducts. Accordingly, use of the term “prosthesis” encompasses the foregoing usages within various types of body passageways, and the use of the terms “intraluminal graft” or “intraluminal medical device” encompasses use for expanding and/or maintaining patency of the lumen of a body passageway. Further, the term “body passageway” encompasses any lumen or duct within the body, such as those previously described, as well as any vein, artery, or blood vessel within the vascular system.

Other vascular applications include anastamosis devices, occlusion devices (for treatment of such disorders as aneurysms or occlusions of blood vessels). Other illustrative applications include treatment of septal defects and closure devices.

Other non-vascular applications include neurological (brain), gastrointestinal, duodenum, biliary ducts, cystic duct, hepatic duct, esophagus, urethra, lymphatic vessels, reproductive tracts, prostate, trachea, and respiratory (such as bronchial) ducts, and otological applications.

Other applications include shunts for various applications, including hydrocephalus, cerebro-spinal fluid shunts, urological applications, glaucoma drain shunts; ear/nose/throat (for example, ear drainage tubes); renal devices; and dialysis (for example, grafts), nerve regeneration conduits, abdominal aortic aneurysm grafts, vascular intervention devices, urinary dilators, circulatory support systems, angiographic catheters, transition sheaths and dilators, tympanostomy vent tubes.

The medical devices of the present invention may be used where the device comes in contact with aqueous systems, such as bodily fluids. Such devices are adapted to release bioactive agent in a prolonged and controlled manner, generally beginning with the initial contact between the device surface and its aqueous environment. The local delivery of combinations of bioactive agents may be utilized to treat a wide variety of conditions utilizing any number of medical devices, or to enhance the function and/or life of the device. Essentially, any type of medical device may be fabricated in some fashion with one or more bioactive agents that enhances treatment over use of the use of the device or bioactive agent.

The devices of the present invention may be used to treat any implantation site within the body in which it is desirable to provide a device that degrades entirely or in part during use. In some embodiments, the device is used to treat an implantation site within the body in which it is desirable to restore and maintain patency or integrity of the implantation site while permitting function of the implantation site. For example, in vascular applications, the device can restore and maintain patency of the vascular site treated with the device, thus permitting continued blood flow through the treatment site. In some embodiments, the inventive device further provides controlled release of one or more bioactive agents.

Devices as disclosed herein may be formed through various methods known to those of skill in the art, including without limitation welding, molding, and winding or braiding of filaments or fibers to form a continuous structure.

In one embodiment, active agents of the present invention are provided via a medical device as described herein. In a preferred embodiment, a part or all of the device is coated with a matrix where the matrix is a polymer or polymers as described herein. In a more preferred embodiment, the matrix includes the drug or active agent. In a most preferred embodiment, the active agent or drug is a cephalotaxine.

The matrices employable for use with drugs or active agents include polymers. The polymers may be biopolymers including without limitation collagen, fibrinogen, hyaluronic acid, lipid complexes, chitins, albumins cyclodextrins, glucosamines, carbohydrate complexes, polylactides, polyglycolides, and copolymers thereof. The polymers may be synthetic polymers including without limitation dacron, nylon, polyurethanes, Lyrca™, Goretex™, polyethylenes, polystyrenes, polypropylenes, polycarbonates, polyethylene glycols, and their copolymers. Other suitable polymers include without limitation poly(L-lactide) (PLLA), poly(D,L-lactide) (PLA), polyglycolide (PGA), poly(L-lactide-co-D,L-lactide) (PLL/PPLA), poly(L-lactide-co-glycolide) (PLLA/PGA), poly(D,L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), poly(hydroxy butyrate), polyacrylate, polyacrylamid, poly(hydroxyethyl methacrylate), elastin polypeptide co-polymer, polyurethane, polysiloxane, ethylene vinyl-acetate, polyethylene terephtalate, thermoplastic elastomers, polyvinyl chloride, polyolefins, cellulosics, polyamides, polyesters, polysulfones, polytetrafluorethylenes, acrylonitrile butadiene styrene copolymers, acrylics, polylactic acid, polyglycolic acid, polycaprolactone, polylactic acid-polyethylene oxide copolymers, cellulose, polymethylmethacrylate, polyalkylene oxalates, poly(dimethyl siloxane), polycyanoacrylates, polyphosphazenes, ethylene glycol I dimethacrylate, poly(methyl methacrylate), poly(2-hydroxyethyl methacrylate), polytetrafluoroethylene poly(HEMA), polyhydroxyalkanoates, poly(glycolide-lactide) co-polymer, poly(y-caprolactone), poly(y-hydroxybutyrate), polydioxanone, poly(y-ethyl glutamate), polyiminocarbonates, poly(ortho ester), polyanhydrides, alginate, dextran, cofton, and their copolymers or derivatized versions thereof, i.e., polymers which have been modified to include, for example, attachment sites or cross-linking groups, in which the polymers retain their structural integrity while allowing for attachment of cells and molecules, such as proteins, nucleic acids, and the like.

The matrices employable for use with the active agents or drugs may include non-polymeric materials. Examples of non-polymeric materials include without limitation sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆, mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides.

The present invention provides medical devices having a coating that may be composed of a matrix containing one or more polymers. The coating may also contain a matrix including one or more non-polymeric materials. In addition, a matrix containing either a polymer or a non-polymeric material may also have one or more active agents as described herein. In one embodiment, a medical device may have more than one coating. In another embodiment, a coating covers substantially the entire surface of the device. In one embodiment, a coating covers a portion of the device. Additionally, any part of the device having contact with organic liquid may likewise be coated. In a preferred embodiment, at least one surface of the medical device is coated with one or more polymers as described herein. Coated devices having a surface coated with such a polymer may provide localized treatment at an implant site. The coating is applied to the device prior to insertion into a patient using methods well known in the art, including without limitation a solvent evaporation method or a controlled vacuum ultrasonic spray deposition process. The coating method may involve mixing one or more polymers as described herein with an active agent as described herein and a solvent, applying the mixture to the surface of a medical device by dipping or spraying, and drying the medical device to evaporate the solvent and polymer(s). The surface of the medical device will then comprise a thin layer film containing active agent. The drying step may also include evaporating the solvent alone leaving a layer of active agent and polymer(s).

In one embodiment, a first coating is applied to a medical device. The first coating may cover part or all of the device. In another embodiment, a second coating is subsequently applied to a medical device. The second coating may cover a first area of the device that was not previously covered by the first coating. Alternatively, the second coating may cover a second area of the device previously covered by the first coating. In another embodiment, the second coating may cover the first area and the second area. In a most preferred embodiment, the second coating does not contain heparin. Additionally, the present invention provides for the application of more than two coatings to medical devices as described herein.

In another embodiment, a coating includes a polymer and more than one drug agent as described herein. Release of a deposited drug agent may be achieved through diffusion through the polymer-fluid interface and then into the fluid. Also, release may occur via the degradation of polymer(s) through hydrolysis, which erodes the polymer-compound layer, thus releasing both into the fluid.

Each coating can be provided on the surface of a device as described herein in a series of applications. The number of applications may be selected to provide individual coated layers of suitable thickness, as well as a desired total number of multiple coatings, as desired. In such embodiments, the coatings may be the same or different, as desired. In other embodiments, the number of applications can be controlled to provide a desired overall thickness to the polymer coating. Generally, the thickness of the coating is selected so that it does not significantly increase the profile of the device for implantation and use within a patient. The overall thickness of a coating as described herein may be from about 1 μm to about 100 μm.

In one embodiment, a coating on a device may be composed of multiple layers of degradable polymer material, each individual layer, or groupings of layers, can include different active agents. For example, in a coronary stent, a coating may include an anti-thrombogenic agent (such as heparin, coumadin and the like) to mitigate acute thrombosis. A coating may also contain an anti-proliferation agent to prevent sub-acute restenosis (for example, a cephalotaxine, everolimus, sirolimus, angiopeptin, paclitaxel, and the like). The coating may contain an anti-inflammatory agent (such as a cephalotaxine, aspirin, lipid lowering statins, fat lowering lipostabil, estrogen and progestin, endothelin receptor antagonist, interleukin-6 antagonist, monoclonal antibodies to VCAM or ICAM, and the like).

In one embodiment, the medical devices of the present invention may be used in the area of cardiovascular medicine. Coronary angioplasty is a medical procedure used to restore blood flow through a narrowed or blocked artery in the heart. The arteries of the heart (the coronary arteries) can become narrowed and blocked due to buildup of a material called plaque on their inner walls. This narrowing reduces the flow of blood through the artery and can lead, over time, to coronary heart disease and heart attack. In angioplasty, a thin tube with a balloon or other device on the end may be first threaded through a blood vessel in the arm or groin (upper thigh) up to the site of a narrowing or blockage in a coronary artery. Once in place, the balloon may then be inflated to push the plaque outward against the wall of the artery, widening the artery and restoring the flow of blood through it. Angioplasty may be used to relieve chest pain caused by reduced blood flow to the heart and/or minimize damage to the heart muscle during a heart attack.

In a most preferred embodiment, the medical device is a stent. Stenosis means constriction or narrowing. A coronary artery that's constricted or narrowed is called stenosed. Buildup of fat, cholesterol and other substances over time may clog the artery. One way to widen a coronary artery is by using PTCA (balloon angioplasty). Some patients who undergo PTCA have restenosis (renarrowing) of the widened segment within about six months of the procedure. Restenosed arteries may have to undergo another angioplasty. One way to help prevent restenosis is by using stents. A stent is a tube that may be composed of metal or plastic and may have either solid walls or mesh walls. Stents may be balloon-expandable or self-expanding. They may be used to prop open an artery after angioplasty.

Stents can be tiny mesh tubes that resemble a small spring and have been used in more recently developed angioplasty procedures. A stent may include a mesh body containing a series apertures. A stent of the present invention may be a coronary stent or a non-coronary stent. The stent may be inserted in the area where the blood vessel, such as an artery, is narrowed to keep it open. Some stents may be coated with medication to help prevent the vessel from closing again. Stents may be used in most angioplasties, where the vessel is large enough to accommodate them. In one embodiment, the stent may be used following an angioplasty procedure to inhibit restenosis by holding open the affected vessel.

Restenosis or the renarrowing of a blood vessel after an angioplasty procedure is less common in stented arteries. Studies are under way using stents covered with drugs that show promise for improving the long-term success of this procedure. Stenosis can also occur after a coronary artery bypass graft (CABG) operation. This type of heart surgery is done to reroute, or “bypass,” blood around clogged arteries. It also improves the supply of blood and oxygen to the heart. In this case, the stenosis may occur in the transplanted blood vessel segments. Like other stenosed arteries, they may need angioplasty or atherectomy to reopen them.

In one embodiment, the devices of the present invention include a restenosis-inhibiting agent. In a preferred embodiment, the device is a stent. Restenosis-inhibiting agents may include a microtubule stabilizing agent such as Taxol, paclitaxel, analogues, derivatives, and mixtures thereof. For example, suitable derivatives include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt. In addition, the restenosis-inhibiting agent may be a cephalotaxine and analogs, derivatives, and mixtures thereof. The inhibiting agent may be dissolved or dispersed in the polymeric materials and the polymeric materials adhered to the stent body. In other embodiments, a matrix as described herein can be sprayed, dipped or extruded onto the stent.

In one embodiment, a coating as described herein is substantially continuous over the stent body. In another embodiment, the coating is primarily over the stent structure but not over the apertures. For example, in a stent formed of a wire mesh, the coating can closely adhere to the wires without covering the apertures therebetween.

A stent according to the present invention can be selected according to desired release dosage profile and provided to the treating physician. After an angioplasty procedure, the coated stent having the restenosis-inhibiting active agent can be delivered to the stenosed, recently dilated coronary artery region. Delivery can be accomplished using methods well known to those skilled in the art, such as mounting the stent on an inflatable balloon disposed at the distal end of a catheter. With the stent advanced into position near the dilated region, the stent can be forced outward and into position against the inner vessel walls. If the stent is self-expanding, the stent can be delivered by deploying the stent from within a delivery device, allowing the stent to expand against the inner vessel walls. The active agent or drug, as it is released from the eroding polymeric coating, can be absorbed by the inner vessel walls. Over time, the polymeric coating is eroded by bodily fluids.

In one embodiment, a medical device as described herein is a drug-delivery device. In a preferred embodiment, this device has at least one surface comprising a coating that includes a polymer as described herein and a compound of the present invention.

In another embodiment, the medical device is a catheter.

The dosage of the compound will depend on the condition being treated, the particular compound, and other clinical factors such as weight and condition of the human or animal and the route of administration or delivery of the compound. It is to be understood that the present invention has application for both human and veterinary use.

In one embodiment of the invention, the cephalotaxine is administered or delivered to a host in the range of 0.05-5.0 mg/m². In a preferred embodiment, the cephalotaxine is administered or delivered to a host in the range of 0.1 to 3.0 mg/m². In a further preferred embodiment, the cephalotaxine is administered or delivered to a host in the range of 0.1-1.0 mg/m².

The cephalotaxine may be administered or delivered biweekly, weekly, daily, twice daily, or more frequently as required to inhibit angiogenesis or to inhibit the onset or progression of an angiogenic disease.

The medical devices can be administered by oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration or delivery. The medical devices may conveniently be presented in unit dosage form and may be prepared by conventional pharmaceutical techniques. Such techniques include the step of bringing into association the medical device and pharmaceutical carrier(s) or excipient(s). In general, the formulations are prepared by uniformly and intimately bringing into association the matrix comprising cephalotaxine with liquid carriers or finely divided solid carriers or both, and then, if necessary, shaping the product.

Formulations of the present invention suitable for oral administration or delivery are usually made from a medical device comprising a matrix containing cephalotaxine where the matrix comprising cephalotaxine is incorporated into capsules, cachets or tablets each containing a predetermined amount of the active ingredient; as a powder or granules; as a solution or a suspension in an aqueous liquid or a non-aqueous liquid; or as an oil-in-water.liquid emulsion or a water-in-oil emulsion and as a bolus, etc.

A tablet may be made by compression or molding, optionally with one or more accessory ingredients. Compressed tablets may be prepared by compressing, in a suitable machine, the matrix comprising cephalotaxine in a free-flowing form such as a powder or granules, optionally mixed with a binder, lubricant, inert diluent, preservative, surface active or dispersing agent. Molded tablets may be made by molding, in a suitable machine, a mixture of the powdered matrix moistened with an inert liquid diluent. The tablets may be optionally coated or scored and may be formulated so as to provide a slow or controlled release of the matrix therein for minutes to hours to days.

Formulations suitable for topical administration or delivery in the mouth include lozenges comprising the matrix comprising cephalotaxine in a flavored basis, usually sucrose and acacia or tragacanth; pastilles comprising the active ingredient in an inert basis such as gelatin and glycerin, or sucrose and acacia.

Formulations suitable for topical administration or delivery to the skin may be presented as ointments, creams, gels and pastes comprising the matrix comprising cephalotaxine to be administered or delivered in a pharmaceutical acceptable carrier. A preferred topical delivery system is a transdermal patch containing the medical device (matrix) to be administered or delivered.

Formulations for rectal administration or delivery may be presented as a suppository with a suitable base comprising, for example, cocoa butter or a salicylate.

Formulations suitable for nasal administration or delivery of the matrix comprising cephalotaxine, wherein the carrier is the matrix or a solid containing the matrix, include a coarse powder having a particle size, for example, in the range of 20 to 500 microns which is administered or delivered in the manner in which snuff is administered or delivered, i.e., by rapid inhalation through the nasal passage from a container of the powder held close up to the nose. Suitable formulations, wherein the carrier is a liquid, for administration or delivery, as for example, a nasal spray or as nasal drops, include aqueous or oily solutions of the active matrix or solid containing the matrix.

Formulations suitable for vaginal administration or delivery may be presented as pessaries, tamports, creams, gels, pastes, foams or spray formulations containing in addition to the medical device such carriers as are known in the art to be appropriate.

Formulations suitable for parenteral administration or delivery include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered or delivered ingredient.

It should be understood that in addition to the ingredients, particularly mentioned above, the formulations of the present invention may include other agents conventional in the art having regard to the type of formulation in question, for example, those suitable for oral administration or delivery may include flavoring agents

Additionally, the cephalotaxine composition of the invention may be administered or delivered with other active compounds. Examples of active compounds that may be co-administered or co-delivered with the cephalotaxine composition include, but are not limited to, other antiangiogenic agents such as angiostatins, VEGF inhibitors, endostatins, combretastatins, 2-methoxy-estradiol, thalidomide and Avastatin™, taxanes, antimetabolites such as methotrexate, corticosteroids, colchicine and analogs, antibodies against angiogenic targets, interferon, diabetic regulating agents such as insulin and insulin growth factor inhibitors, anti-inflammatory agents such as COX-2 inhibitors, anti-arthritics, aspirin, ibuprofen, naprosyn and the like, gene therapy, antisense therapy, and RNA interference therapy against gene targets and associated mRNA and protein targets of angiogenesis, antisense therapy, and RNA interference therapy.

The active ingredient may administered or delivered to the host before, during or after administration of the cephalotaxine composition. In one embodiment of the invention, the active ingredient is mixed with the cephalotaxine prior to administration and the mixture is administered or delivered to the host. In a further embodiment, the active ingredient and the cephalotaxine are administered or delivered separately but simultaneously to the host. In yet a further embodiment, the active ingredient is administered or delivered before the cephalotaxine. In a preferred embodiment, the active ingredient is administered or delivered before the cephalotaxine with the active ingredient still present systemically in the host. In yet a further embodiment, the active ingredient is administered or delivered after the cephalotaxine. In a preferred embodiment, the active ingredient is administered or delivered after the cephalotaxine while the cephalotaxine is still present systemically in the host.

Suitable hosts of the invention include humans or other animals.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are hereby expressly incorporated by reference.

### EXAMPLES

### Example 1

### Effects of Homoharringtonine in the CAM Assay

Protocol:

Fertilized chicken eggs (HiChick Breeding Co, Kapunda, South Australia) were incubated for three days at 38° C. On Day 3 the embryos were cracked out of the egg and into a cup made of plastic piping, with plastic film stretched over the top to form a hammock for the egg to be suspended in. Two ml of DMEM containing penicillin and streptomycin was added to each cup prior to the egg being added. A Petri dish on the top maintained sterility. Incubation continued in a humidified 37° C. incubator.

On Day 4 the chorioallantoic membrane (CAM) begins to grow, and pictures were taken of each embryo at ×5 to measure the CAM area using image analysis software (Video Pro 32, Leading Edge Pty Ltd, South Australia). Embryos were then grouped according to their CAM area, with a control embryo in each for comparison. There were four matched embryos, treated with 6.25, 12.5 and 25 ng of homoharringtonine. Grouping is critical as in these early developmental stages changes in the CAM growth are dramatic. Relatively small differences in size on Day 4 translate to large differences in the CAM on Day 5, making it impossible to compare treatments. Substances were applied in methylcellulose discs, which were first dried under vacuum overnight. The methylcellulose discs were applied to the top of the CAM, and at the beginning of treatment were at least three to four-fold bigger than the CAM area, meaning treatment covered the entire CAM surface.

On Day 5 skim milk with contrast medium was injected into the CAM. Pictures were then taken at various levels of magnification up to ×63. Quantitative measurements were made from ×5 pictures. CAM area, and vein and artery lengths were measured using image analysis (Video Pro 32, Leading Edge Pty Ltd, South Australia). Relative vessel lengths were then calculated as the total length/CAM area. Statistical analysis was made using SigmaStat and OneWay ANOVA with p<0.05 as the level of significance.

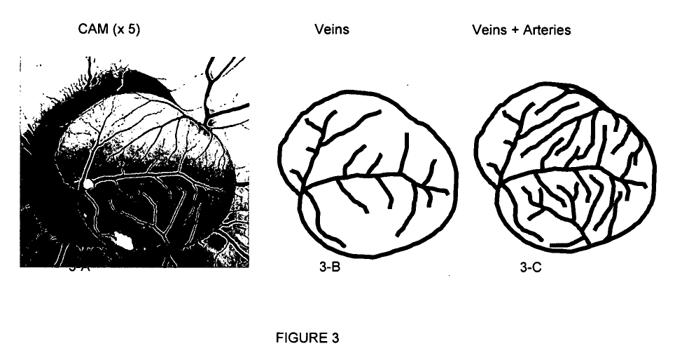

FIG. 3A illustrates the normal organization of the CAM is uniform, with the major vein draining towards the left, and the artery branches coming over the edge of the top and bottom of the CAM. FIGS. 3B and 3C schematically illustrate tracing of the vein and artery branches, as performed for the measurement of vessel lengths.

The angiogenic inhibitor, homoharringtonine, was obtained from ChemGenex Therapeutics, Inc. (Menlo Park, Calif.) and was made to the appropriate concentration in sterile water. At the initial doses that were used homoharringtonine resulted in the death of the embryos, hence the dose was reduced. Homoharringtonine was applied at 6.25, 12.5 and 25 ng (11.3, 22.5 and 45 nM) doses, and compared with water treated controls. Results are shown in Table 2. Homoharringtonine reduced the growth of the CAM to 42% of the control in the 25 ng treated CAM. The vein, artery and total vessel lengths were also significantly reduced in the 25 ng group, with non significantly reduced vessel lengths in the 6.25 and 12.5 ng treated groups. The vein, artery and total vessel lengths were reduced to 15%, 18% and 17% of control, respectively. Not surprisingly the relative vessel lengths were also reduced, with the relative artery lengths being significantly reduced at all three dose levels of homoharringtonine, and the relative vein and total vessel lengths significantly different only at the highest dose of homoharringtonine.

TABLE 2
Homoharringtonine (6.25, 12.5 and 25 ng versus DMSO control; Mean +/− SEM)

| | | | |
|---|---|---|---|
| Water | 6.25 ng | 12.5 ng | 25 ng |
| n = 6 | n = 6 | n = 6 | n = 6 |

CAM area (pixels)

| | | | | |
|---|---|---|---|---|
| Day 4 | 6.1 ± 1.4 | 6.5 ± 1.6 | 6.3 ± 1.6 | 6.2 ± 1.5 |
| Day 5 | 65.3 ± 18.3 | 45.3 ± 11.6 | 53.0 ± 11.6 | 30.2 ± 9.9 |
| CAM increase (fold) | 10.2 ± 0.8 | 7.0 ± 0.4 ^{a} | 9.0 ± 1.0 ^{b} | 4.3 ± 0.7 ^{a} |

Vessel lengths (pixels)

| | | | | |
|---|---|---|---|---|
| Vein length | 2382 ± 717 | 1482 ± 499 | 1564 ± 427 | 359 ± 143 ^{a} |
| Artery length | 3009 ± 884 | 1573 ± 516 | 1787 ± 544 | 551 ± 265 ^{a} |
| Total vessel length | 5391 ± 1596 | 3055 ± 1003 | 3351 ± 953 | 909 ± 396 ^{a} |

Relative vessel lengths (length/CAM area)

| | | | | |
|---|---|---|---|---|
| Relative vein length | 36.2 ± 4.9 | 31.6 ± 4.6 ^{b} | 28.3 ± 4.4 ^{b} | 11.3 ± 3.5 ^{a} |
| Relative artery length | 45.1 ± 1.6 | 31.6 ± 4.5 ^{a b} | 31.8 ± 3.7 ^{a b} | 13.0 ± 4.3 ^{a} |
| Relative total vessel length | 81.4 ± 6.1 | 63.2 ± 7.7 ^{b} | 60.1 ± 7.5 ^{b} | 24.3 ± 6.7 ^{a} |

^{a} p < 0.05 vs control;
^{b} p < 0.05 vs 25 ng

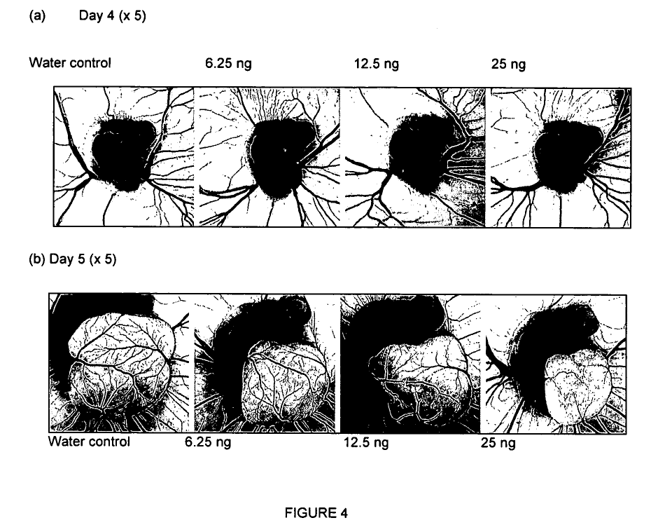

Homoharringtonine treatment of the CAMs resulted in a significant reduction in blood vessels, as illustrated in FIG. 4.

As seen in FIG. 4, even at the lowest dose of homoharringtonine the CAM is smaller and the normal vessel organization disturbed. Note the overlaying of a major vein and artery branch at the bottom of the CAM. The CAM at 12.5 ng has a general reduction in vessels without a great deal of disturbance in the organization. The highest dose of 25 ng resulted in only fine vestigial blood vessels remaining, and blood vessel development almost completely blocked. The 25 ng dose killed one of the smaller embryos.

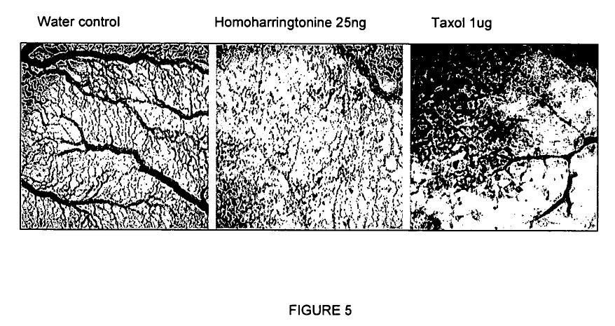

The changes seen due to homoharringtonine at higher magnifications were unique, and unlike other substances that have been tested. In FIG. 5 a normal CAM and 25 ng homoharringtonine treated CAM are shown. The water control is well vascularized. Homoharringtonine treatment has resulted in a dramatic reduction in blood flow, with only a few fine vessels in the field carrying red blood cells. The unique feature is the black dots spread through the field of view representing red blood cells that have been trapped in blood vessels in which flow has ceased. Compare this to the changes seen with taxol, with diffuse leakage of the red blood cells outside the vessels and the skeletons of larger vessels with no remaining blood flow.

The antiangiogenic activity of homoharringtonine was tested using the early chicken chorioallantoic membrane (CAM). The use of homoharringtonine resulted in significant reductions in blood vessel development in the CAM, with differences in both the potency and the qualitative changes observed from that of taxol. These differences may reflect varying mechanisms of action, such as affecting endothelial cell proliferation, apoptosis, and migration due to these substances.

What is claimed is:

1. A medical device comprising a device body and a coating on a surface of said device body, wherein said coating comprises a cephalotaxine.

2. The medical device of claim 1 wherein said medical device is other than a stent.

3. The medical device of claim 1 wherein said device is selected from the group consisting of catheters, endoscopes, wound healing dressings, tissue/organ barriers, sutures, artificial organs, artificial organoids, implantable monitors, defibrillators, pacemakers, implantable pumps, cell reservoirs, prosthetic devices, and orthopedic devices.

4. A medical device comprising
a stent; and
a coating on a surface of said stent comprising a cephalotaxine, wherein said device does not have an additional single coating on said stent containing heparin.

5. The medical device of claim 1 or 4 wherein said coating further comprises a polymer.

6. The medical device of claim 5 wherein said polymer comprises a biopolymer.

7. The medical device of claim 6 wherein said biopolymer is selected from the group consisting of collagen, fibrinogen, hyaluronic acid, lipid complexes, chitins, albumins cyclodextrins, glucosamines, carbohydrate complexes, polylactides, polyglycolides, and copolymers.

8. The medical device of claim 5 wherein said polymer comprises a synthetic polymer.

9. The medical device of claim 8 wherein said synthetic polymer is selected from the group consisting of dacron, nylon, polyurethanes, Lyrca™, Goretex™, polyethylenes, polystyrenes, polypropylenes, polycarbonates, and polyethylene glycols.

10. The medical device of claim 1 or 4 wherein said coating further comprises a non-polymeric material.

11. The medical device of claim 1 or 4 wherein said cephalotaxine comprises homoharringtonine (cephalotaxine, 4-methyl-2-hydroxy-2-(4-hydroxy-4-methyl pentyl) butanediocate ester).

12. The medical device of claim 1 or 4 wherein said cephalotaxine comprises a compound of the formula 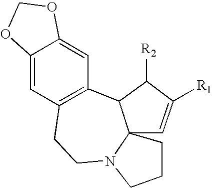
wherein R₁ is an ester or a substituted alkyl and wherein R₂ is an ester or a substituted alkyl.

13. The medical device of claim 1 or 4 wherein said device further comprises a second coating.

14. The medical device of claim 1 or 4 wherein said device comprises more than two coatings.

15. The medical device of claim 1 or 4 wherein said coating further comprises a second agent.

16. A medical device comprising a matrix and a cephalotaxine contained therein.

17. The medical device of claim 16 wherein said matrix is biodegradable.

18. The medical device of claim 16 wherein said matrix is a time release matrix.

19. A method comprising contacting a host with the medical device of claim 1, 4, or 16 in vivo, wherein said device provides a sufficient amount of said cephalotaxine to inhibit angiogenesis.

20. A method of treating an angiogenic disease in a host comprising contacting said host with the medical device of claim 1, 4, or 16 in vivo, wherein said device provides a sufficient amount of said cephalotaxine to inhibit the onset or progression of an angiogenic disease.

21. The method of claim 19 or 20 wherein said contacting step comprises implanting said medical device in said host.

22. A delivery device comprising a catheter having a lumen and the device of claim 4 contained within said lumen.
